(19) United States
(12) Patent Application Publication
(10) Pub. No.: US 2024/0004421 A1
(43) Pub. Date: Jan. 4, 2024

(54) MULTI-MEDIA INSERT TREATMENT AND TRAINING AID

(71) Applicant: Wessely Joesph Murphy, Barboursville, WV (US)

(72) Inventor: Wessely Joesph Murphy, Barboursville, WV (US)

(21) Appl. No.: 18/112,080

(22) Filed: Feb. 21, 2023

Related U.S. Application Data
(60) Provisional application No. 63/312,372, filed on Feb. 21, 2022.

(51) Int. Cl. G06F 1/16; H04N 21/81; A61F 17/00

(52) U.S. Cl. CPC G06F 1/1605; H04N 21/8106; G06F 1/1635; A61F 17/00

(57) ABSTRACT

A multi-media insert that displays videos to a user relating to the training, treatment, or quick response measures for a particular condition is described. The multi-media insert is designed to help individuals respond or treat a condition presented on a subject by providing audio and visual training and treatment information to the individual to aid or guide the individual in responding or treating the subject. In certain embodiments the multi-media insert fits within a container such as a first aid box or other container that hold various treatment supplies related to the treatment of the condition and makes audio visual training and instruction in responding or treating a condition immediately available along with the various treatment supplies. Having the multi-media insert readily available with the treatment supplies can potentially remove stress and avoid common mistakes in a treatment situation by guiding the individual through the response and treatment process.

### CROSS-REFERENCE TO RELATED APPLICATIONS

The present application claims prior to U.S. Provisional Patent Application No. 63/312,372, filed on Feb. 21, 2022, specifically incorporated herein by reference in its entirety.

### FIELD OF THE INVENTION

The present invention is generally directed to a device that provides instructions on the treatment of a condition. More particularly, the present invention is directed to multi-media insert that fits within a container such as a first aid container and provides audio visual treatment guidance on a condition.

### SUMMARY OF THE INVENTION

Certain embodiments of the present invention may include multi-media insert comprising a video monitor, a speaker, a switch, a video controller having video file storage memory, and a video file including video content and audio content having condition specific information for a specific condition stored in the video file storage memory. The video controller is in communication with the video monitor, the speaker, and the switch, and the video controller plays the video content on the video monitor and plays the audio content on the speaker when the switch is activated.

Some embodiments may include a cover substantially covering the video monitor, wherein the cover is in communication with the switch and activates the switch when the cover is moved away from the video monitor. The cover may be connected to the multi-media insert by a hinge, wherein the cover substantially covers the video monitor in a closed position and provides visual access to the video monitor when the cover is in the open position, and wherein the cover activates the switch when the cover is moved towards the open position.

In some embodiments the switch may be a button-type switch, optical switch, magnetic switch, pressure switch, light sensor, motion sensor, virtual switch on a touchscreen monitor, or a hinge sensor.

In further embodiments, the multi-media insert may include a plurality of video files stored in the video file storage memory, wherein each of the plurality of video files includes video image content and video audio content having condition specific information for a specific condition, wherein each of the plurality of video files contain different condition specific information; and a plurality of switches in communication with the video controller, wherein at least one of the plurality of switches is programmed for the video controller to play at least one of the plurality of video files and provide the video image content to the video monitor and the audio content to the speaker when the at least one of the plurality of switches is activated.

In some embodiments, a switch may control playback functions of the video file and audio content may contain audio content in more than one language.

In additional embodiments, the audio content contains audio content in at least two languages, and one of the plurality of switches is programmed to select one of the at least two languages when the at least one video of the plurality of videos is played on the video monitor and the speaker.

In some embodiments, one video file of the plurality of video files may contain condition specific information for a first condition, and a second video file of the plurality of video files may contain condition specific information for a second condition, wherein one switch of the plurality of switches is programmed for the video controller to play the one video file and a second switch of the plurality of switches is programmed for the video controller to play the second video file. In some embodiments the first condition and second condition are different.

In certain embodiments, a condition may include one or more of the following: drug overdoses, opioid overdose, snake bites, allergic reactions, choking, CPR, cardiac arrest, heat related illnesses, burns, lacerations, bone fractures, disposal of medications, basic first aid, eye injuries, dental injuries, biohazardous spills, drowning and near drowning conditions, blood borne pathogen incidents, pet first aid, and food related illness.

Further, some embodiment of the multi-media insert may include a code scannable by a mobile device that retrieves and plays an internet-based video file that has substantially the same video content and substantially the same audio content as the video file stored in the video file storage memory.

Still further, the multi-media insert may include at least one near field communication chip, wherein the near field communication chip is programmed for a mobile device to dial emergency services.

In yet further embodiments, the multi-media insert may include at least one near field communication chip, wherein the near field communication chip is programmed for a mobile device to retrieve and play an internet-based video file that has substantially the same video content and substantially the same audio content as the video file stored in the video file storage memory.

In certain embodiments, the multi-media insert is sized to fit in a lid of a container or plastic container.

The multi-media insert may include a power supply connected to the video controller and provides electrical power to the multi-media insert to run the video controller, video monitor, and speaker. In some embodiments, the power supply may be a rechargeable battery.

Additional embodiments of the invention may include a treatment kit comprising a container having a lid, a treatment materials housed inside the container; and a multi-media insert positioned on lid and within the container, the multi-media insert comprising, a video monitor, a speaker, a switch, a video controller having video file storage memory, and a video file including video content and audio content having condition specific information for a specific condition stored in the video file storage memory, wherein the video controller is in communication with the video monitor, the speaker, and the switch, and wherein the video controller plays the video content of the on the video monitor and plays the audio content on the speaker when the switch is activated.

### BRIEF DESCRIPTION OF THE DRAWINGS

The importance of the features and merits of the present invention will be better understood by reference to the attached drawings. It is to be understood, however, that the drawings are designed for the purpose of illustration only and not as a definition of the limits of the present invention.

FIG. 1 is a diagrammatic view of a multi-media insert in accordance with an embodiment of the invention.

FIG. 2 is a diagrammatic view showing components of a multi-media insert in accordance with an embodiment of the invention.

FIG. 3 is a diagrammatic view of a multi-media insert in accordance with another embodiment of the invention.

FIG. 4 is a diagrammatic view of a container in accordance with certain embodiments of the invention.

FIG. 5 is a diagrammatic view of a treatment kit in accordance with an embodiment of the invention.

FIG. 6 is a diagrammatic view of a treatment kit in accordance with another embodiment of the invention.

### DETAILED DESCRIPTION OF EMBODIMENTS OF THE INVENTION

Some preferred embodiments of the present invention are described in this section in detail sufficient for one skilled in the art to practice the present invention without undue experimentation. It is to be understood, however, that the fact that a limited number of preferred embodiments are described in this section does not in any way limit the scope of the present invention as set forth in the claims.

It is to be understood that whenever a range of values is described herein, i.e. whether in this section or any other part of this patent document, that the range includes the end points and every point therebetween as if each and every such point had been expressly described. Unless otherwise stated, the words “about” and “substantially” as used herein are to be construed as meaning the normal measuring and/or fabrication limitations related to the value or condition which the word “about” or “substantially” modifies. Unless expressly stated otherwise, the term “embodiment” is used herein to mean an embodiment of the present invention.

First aid kits and other treatment related kits provide materials for the treatment of certain conditions requiring aid. However, the user must have a certain amount of knowledge about the use of the materials to effectively treat the condition. Sometimes a first aid kit or treatment related kit may include written instructions or a manual for someone to read and refer to for information on how to treat a particular condition. When time is of the essence or a user is under stress, the user may not have the time to locate or read the written materials. This may inhibit the ability of the user to provide effective treatment.

The present invention addresses these issues by providing a multi-media insert that displays videos to the user relating to the training, treatment, or quick response for a particular condition. The multi-media insert is designed to help individuals respond or treat a condition presented on a subject by providing audio and visual training and treatment information to the individual to aid or guide the individual in responding or treating the subject. In certain embodiments the multi-media insert fits within a container such as a first aid box or other container that hold various treatment supplies related to the treatment of the condition and makes audio visual training and instruction in responding or treating a condition immediately available along with the various treatment supplies. Having the multi-media insert readily available with the treatment supplies can potentially remove stress and avoid common mistakes in a treatment situation by guiding the individual through the response and treatment process.

The multi-media insert is configured to provide information pertaining to the response or treatment of certain conditions of a subject in the form of videos that are viewable by a user. Conditions of a subject may include, but is not limited to drug overdoses, such as opioid overdose, snake bites, allergic reactions, choking, CPR, cardiac arrest, heat related illnesses, burns, lacerations, bone fractures, disposal of medications, basic first aid, eye injuries, dental injuries, biohazardous spills, drowning and near drowning conditions, blood borne pathogen incidents, pet first aid, and food related illness and other conditions requiring some form of treatment or bystander intervention.

The information provided to the user by the multi-media insert is referred to as condition specific information and may include, but is not limited to, training information, treatment method information, and emergency or quick response information for a specific condition. Training information is a video having audio and video content that is intended for training a user on responding or treating a specific condition. Training information videos are typically longer and more thorough than the treatment method information videos or emergency or quick response information videos. The treatment method information are videos having audio and video content directed to the method for treating a specific condition. The emergency or quick response video is a relatively concise video having audio and video content directed to the steps the user needs to take to respond or treat a specific condition. As will be discussed below the multi-media insert may be configured to provide condition specific information for a single condition or, in other embodiments, may be configured to provide condition specific information for multiple conditions.

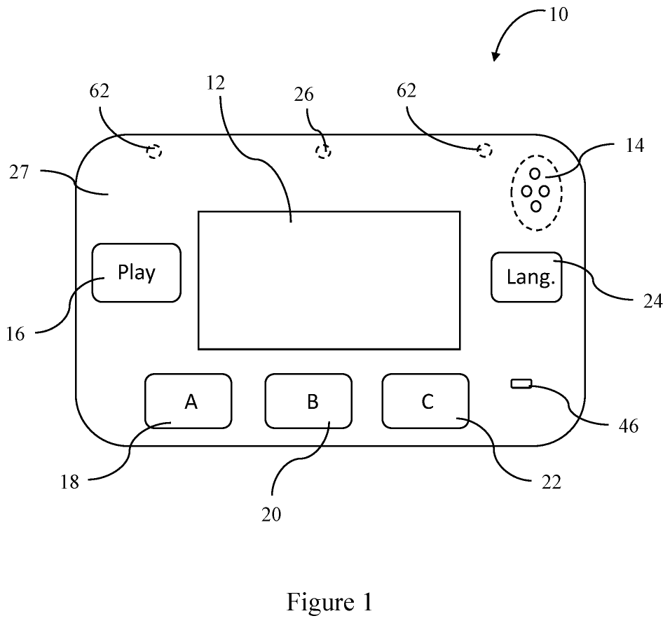

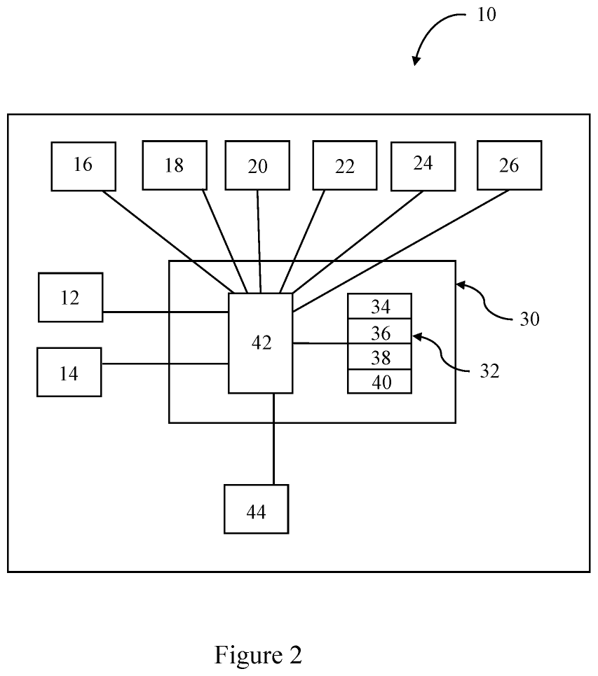

With reference now to FIGS. 1 and 2, there is illustrated a multi-media insert 10 in accordance with an embodiment of the invention. The multi-media insert 10 includes a video monitor 12, a speaker 14, and at least one switch or a plurality of switches represented by reference numerals 16, 18, 20, 22, 24, and 26 that control the playback of one or more video files that contain condition specific information, which will be discussed in detail below.

The video monitor 12 displays video content from one or more video files stored on the multi-media insert 10. The size and resolution of the video monitor is not particularly limited but should be large enough an individual is able to readily view the video content. The video monitor is preferable able to display videos in all the common video aspect ratios, including but not limited to 3:2, 4:3, 5:4, 16:10, 16:9, 1.85:1, and 2.35:1. The resolution of the video monitor may include, but is not limited to, standard definition, high definition, full high definition, 2k, 4k, and 8k. The size of the video monitor is measured diagonally (the distance from one corner of the video monitor to the diagonal corner on the video monitor). In certain embodiments the size of the video monitor may range from about 4 inches to about 12 inches. Preferably, the size of the video monitor ranges from about 4 inches to about 6 inches. The thickness of the video monitor is not crucial but is preferably as thin as possible to reduce the overall size of the multi-media insert 10 as much as possible. In certain embodiments the thickness of the video monitor may be less than about 0.25 inches. In some embodiments, the video monitor 12 may be a touchscreen video monitor where virtual switches may be programmed as user selectable buttons on the touchscreen video monitor.

The video monitor 12 is connected to a video controller 30. The video controller 30 includes video file storage memory 32. At least one or a plurality of video files, represented by the reference numerals 34, 36, 38, and 40 are stored in the video file storage memory. The video files 34, 36, 38, and 40 each include video content and audio content that contains condition specific information. In some embodiments, at least two of the plurality of video files contain different condition specific information. For example, one of the video files may include condition specific information for a first condition and a second video file may contain condition specific information for a second condition where the first condition is different than the second condition. In additional embodiments, one of the plurality of video files may contain condition specific information that is training information, while a second video of the plurality of video files contains condition specific information that is emergency or quick response information. As discussed above condition specific information are videos having audio content and video content directed to a specific condition and may include training information, treatment method information, or emergency or quick response information. In some embodiments, the plurality of video files may pertain to providing instruction and training on responding and treating an opioid overdose. In other embodiments, the plurality of video files may separately pertain to separate conditions such as instruction and training on the treatment of lacerations, burns, and fractures.

The format of the plurality of video files is not limited and may include any of the common video formats known to those skilled in the art, including but not limited to, MP4, MOV, WMV, AVI, AVCHD, WEBM, HTML5, or other known video format. The video format of the plurality of video files should be a format that is compatible with the video controller 30 and video processor 42. The audio content of the video file may include audio content in more than one language.

While the embodiment represented in FIGS. 1 and 2 illustrate four video files, the number of video files is not particularly limited. Certain embodiment of the invention may have only one video file, while other embodiments of the invention may include at least two video files. Still further, embodiments of the invention may include about 2 to about 30 video files, or more. The number of video files is only limited by the size of the video file storage memory 32. The video file storage memory 32 should be sized appropriately for the desired size and number of video files. In certain embodiments the size of the video file storage memory 32 may range from about 64 MB to about 500 MB, and preferably about 256 MB.

The video controller 30 includes a video processor 42 in communication with the video file storage memory 32. The video processor 42 controls the playback of one or more of the plurality of video files 34, 36, 38, and 40 by rendering video content from the video file and displaying the video content on the video monitor.

The video processor 42 sends the audio content from the video file to the speaker 14. The speaker 14 plays the audio content for the user as the video content is played on the video monitor 12. The size of the speaker 14 is not particularly limited but should be able to play the audio content so a user watching the video content can clearly hear the audio content. In some embodiments the speaker 14 may be a 2 watt, 8 ohm speaker. The speaker should be sized to fit within the multi-media insert.

The multi-media insert 10 may also include a plurality of switches, represented by the reference numerals 16, 18, 20, 22, 24, and 26. While the embodiments illustrated in FIGS. 1 and 2 illustrate six particular switches, some embodiments of the invention may have only one switch. Other embodiments may have at least two switches. In certain embodiments, the number of switches may range from about 1 to about 10 switches. The number of switches is not particularly limited and may vary depending on the particular features needed for the multi-media insert 10.

In some embodiments, each of the plurality of switches 16, 18, 20, 22, 24, and 26 is programmed in conjunction with the video controller 30 to control certain features related to the playback of the plurality of video files. For example, switch 18 is programmed to play one video file from the plurality of video files when activated. Similarly, switch 20 is programmed to play a second video file from the plurality of video files when activated. Further, switch 22 is programmed to play a third video file from the plurality of video files when it is activated. The plurality of switches can be programmed in other ways as well. For example, switch 16 may be programmed to start, pause and stop the playback of the current video, and switch 24 is programmed to change the language of the audio content. Additional switches may be used to control the volume of the audio content played through the speaker 14.

The type of switch used for the plurality switches is not particularly limited and may include, button-type switch, optical switch, magnetic switch, pressure switch, light sensor, motion sensor, hinge sensor, virtual buttons programmed for use on a touchscreen video monitor, or others known to those skilled in the art depending on the application of the switch. Where the individual or user selectable features are needed, the switch is preferably a button type switch they requires the user to depress the button to activate the switch. For example, switches 16, 18, 20, 22, and 24 are preferably button type switches the user depresses to activate the corresponding switch. In an alternative embodiment, the video monitor 12 may be a touchscreen monitor having touchscreen functionality. In this alternative embodiment, physical switches such as pressable buttons that are activated by a user pressing the button may be replaced with electronic switches programmed as virtual buttons on the touchscreen where the user taps the virtual button on the touchscreen to activate the desired video file.

The embodiment in FIG. 1 illustrates a switch that does not require the user to press or select. Switch 26 may be a magnetic switch or an optical switch that is activated when the multi-media insert is removed for use from a container or packaging. When switch 26 is activated, the multi-media insert plays a specified video file from the plurality of video files. This automatic playing of the video file may be advantageous to quickly provide condition specific information to the user as quickly as possible, particularly where the condition specific information is emergency or quick response information.

Programming of the plurality switches may be done by the manufacturer or supplier during the assembly of the multi-media insert. In some embodiments one or more of the plurality of switches may be programmed after assembly. Still further video files may be added or deleted from the video file storage memory after assembly. Programming the switches and the addition or deletion of video files from the video file storage memory may take place through a port 44. In order to protect the video files from corruption or accidental deletion, a lock code in the form of pressing a certain combination of switches, may be utilized to prevent any changes until the lock code is provided.

The multi-media insert 10 also includes a power supply 44. The type of power supply is not particularly limited and may include any of those know to those skilled in the art. In certain embodiments, the power supply 44 includes one or more batteries. In certain embodiments the power supply 44 is a rechargeable battery such as a lithium ion battery. Where the power supply 44 is a rechargeable battery, port 46 is a multiuse port that supports recharging the rechargeable battery. In some embodiments, port 46 may be a micro USB or USB-C port. The capacity of the battery should be large enough to provide electrical power to the video controller, video monitor, and speaker to play back the plurality of video files at least 3 times. In certain embodiments, the capacity of the power supply is suitable to play back the plurality of video files from about 20 to about 100 times or more. In some embodiments, a video file will play and repeat from about 2 to about 5 times. The multi-media insert 10 may include a feature that puts the multi-media insert 10 to sleep once the video file has finished playing the designated number of times or after a certain period of time with no input from the user.

The video monitor 12, the video controller 30, the speaker 14, the plurality of switches 16, 18, 20, 22, 24, and 26 are mounted onto a mounting board. In some embodiments, the mounting board may be a rigid foam board with locations cut out to mount and hold the video monitor 12, the video controller 30, the speaker 14, and one or more of the plurality of switches 16, 18, 20, 22, 24, and 26. The mounting board provide some rigidity and durability to the multi-media insert 10. The mounting board with the video monitor 12, the video controller 30, the speaker 14, the and the plurality of switches 16, 18, 20, 22, 24, and 26 may be covered with a wrap 27. The wrap may be a durable paper, cardboard, or cardstock with an appropriate cut out to reveal the video monitor that is wrapped around and secured to the mounting board. In some embodiments the wrap may be made of polymer or plastic materials and may have impact resistant properties. It may be desirable in some embodiments to provide holes in the wrap around the speaker for sound to escape. In some embodiments the switches 16, 18, 20, 22, and 24 are covered by the wrap. Graphics on the wrap may be provided to indicate where the switches are located, and the wrap is sufficiently pliable to allow for the switch to be depressed and activated by the user. The wrap may include graphics to provide the user with written instructions or information.

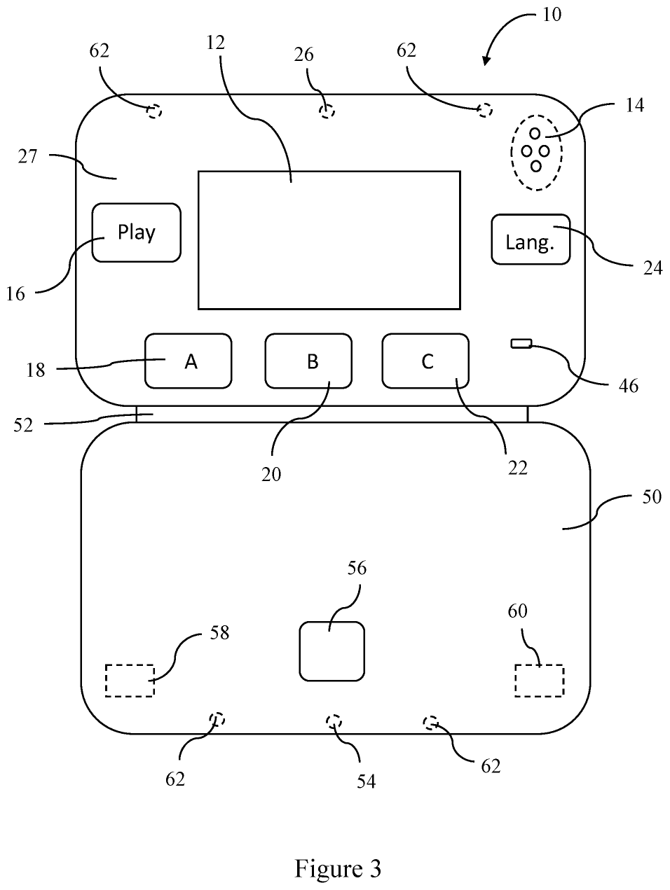

With reference to FIG. 3 there is illustrated another embodiment of the present invention. The embodiment provided in FIG. 3 is the same embodiment discussed in FIG. 1 with additional features. The multi-media 10 may include cover 50. The cover 50 is connected to the multi-media insert 10 by a hinge 52. The cover 50 folds onto the multi-media insert 10 to substantially covers the video monitor 12 in a closed position. The cover 50 is folded away from the multi-media insert 10 to expose the video monitor 12 in an open position. The moveable video monitor cover 50 and hinge 52 may be made from the same material used for the wrap and in some embodiments may be an extension of a portion of the wrap used for the multi-media insert 10.

In some embodiments, the cover 50 may include at least one magnet 52 positioned near the edge furthest from the hinge 52 and is positioned to engage with switch 26 when the cover 50 is in the closed position. When the cover 50 is in the closed position, switch 26 is not activated and the multi-media insert 10 is turned off. When the cover 50 is moved to the open position, the magnet moves away from the magnetic switch 26 and activates switch 26 to play the video file programmed to the activation of switch 26. While this embodiment is described with respect to a magnetic switch for switch 26, other switches such as an optical switch may be used instead, such that the optical switch detects with the cover is in the open position and then plays the video file programmed for the optical switch activation.

The multi-media insert 10 may also include additional features that provide redundancy to the plurality of video files. For example, the multi-media insert 10 may include a scannable code 56, such as a QR code, that is scannable by a mobile device and retrieves and plays an internet-based video file on the mobile device that has substantially the same video content and substantially the same audio content as the plurality of video files stored in the video file storage memory.

In additional embodiments, the multi-media insert 10 may include one or more near field communication chips programmed to provide different functions to a mobile device capable of detecting near field communication chips. Near field communication chips are sometimes referred to as tap chips or tappable chips. In certain embodiments, a first near field communication chip 58 may be programmed for the mobile device to dial emergency services such as 9-1-1. Another near field communication chip 60 may be included and programmed for a mobile device to retrieve and play an internet-based video files on the mobile device that has substantially the same video content and substantially the same audio content as the plurality of video files stored in the video file storage memory.

The use of scannable codes and near field communication chips provides redundancy to provide the user of the multi-media insert 10 the needed condition specific information for the specific condition.

While the near field communication chips and the scannable code are illustrated on the cover 50, they may be placed anywhere on the multi-media insert 10 that provides easy access to the individual or user.

To assist in keeping the cover 50 in a closed position, one or more pairs of magnets 62 may be used. One magnet is placed near the edge of the cover 50 furthest from the hinge 52, and a corresponding magnet (opposite pole) is placed on the edge of the multi-media insert 10 such that the magnets 62 engage when the cover is in a closed position. In some embodiments, the cover 50 includes a pull tab 64 to assist in moving the cover 50 from the closed position to the open position. The pull tab 64 may be located on an edge of the cover 50 or on an outside surface 68 of the cover 50. The outside surface 68 of the cover 50 is that surface which is exposed when the cover 50 is in the closed position. The pull tab 64 may be a ribbon, cut-out, plastic tab, or other similar material attached to the outside surface 68 of the cover 50 allowing for the user to grip the pull tab and facilitate the movement of the cover 50 into the open position.

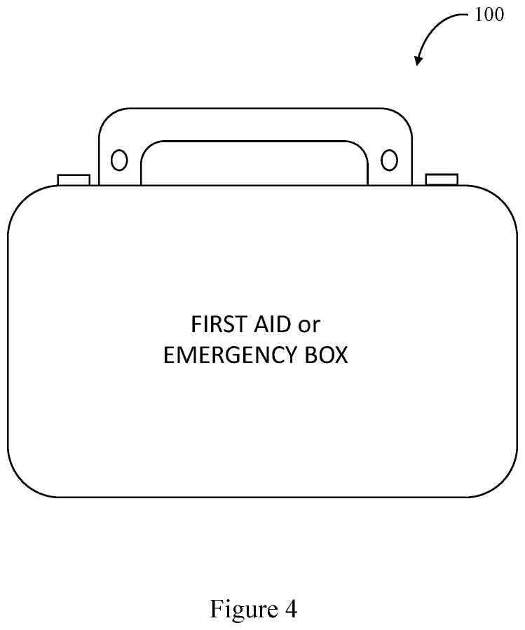

In certain embodiments, the multi-media insert 10 is sized to fit in a pre-selected container that contains treatment materials, such as a first aid container. Some standard size containers that may be suitable may include, but are not limited to 10 unit plastic case (10U), 25 unit plastic case (25U), and 50 unit plastic case (50U) containers commercially available. The size of the container is not particularly limited. In certain embodiments the size of the container may range from about 3 inches to about 24 inches for the height, about 3 inches to about 24 inches for the length, and about 1 inch to about 12 inches for the width/thickness. Preferably the container is made from plastic. In some embodiments the lid for the container is attached to the body of the container. In some embodiments, the container may be water resistant or weather resistant. FIG. 4 illustrates an example of a certain 10U container 100 designed as a container first aid material.

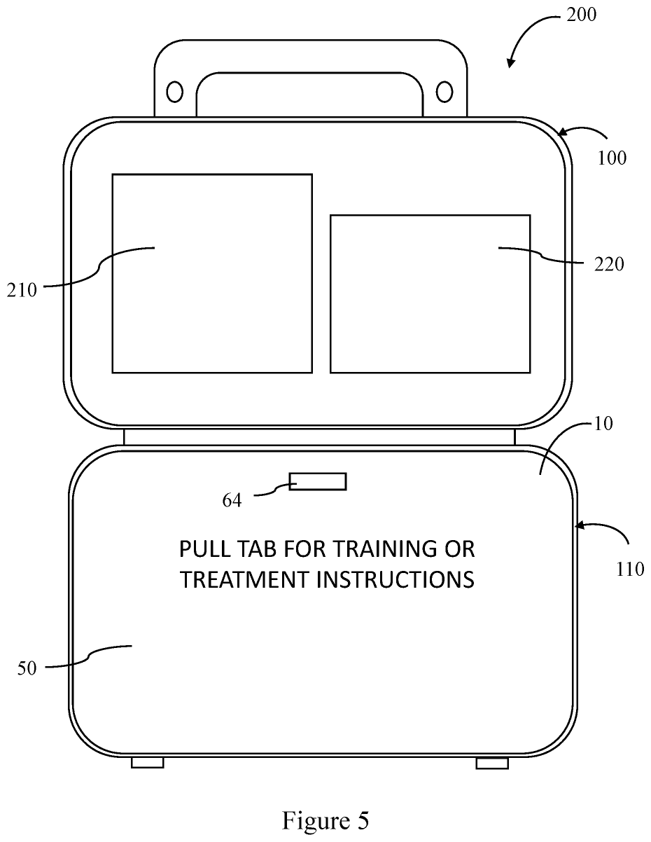

In further embodiments, the multi-media insert 10 is sized to fit within the inside of the lid of a container 100. With reference to FIG. 5, there is illustrated a container 100 with the lid 100 open. The multi-media insert 10 is positioned within the inside of the lid 110. Preferably the multi-media insert 10 is securely fastened to the inside of the lid 110 such as by glue or double sided tape. Alternatively, the multi-media insert 10 may be removeably fastened to the inside of the lid 110 using hook and loop type fasteners or other similar fasteners.

FIG. 5 illustrates a treatment kit 200 in accordance with an embodiment of the invention. The treatment kit includes a container 100 and a multi-media insert 10 sized to be positioned on the lid 110 within the container and personal protective equipment 210 and treatment materials 220. The cover 50 of the multi-media insert 10 is in the closed position, exposing the pull tab 64. The treatment materials 210 and 220 are materials for use in treating the condition the treatment kit is designed to treat. Personal protective equipment 210 may include those items to protect the user during treatment of the specified condition. In some embodiments personal protective equipment 210 may include one or more of gloves, masks, aprons, protective clothing, respirator, eye protection, sanitary wipes, or other additional protective items based on the particular condition the kit is designed to address. Treatment materials 220 may include specific or specialized items needed for treating the desired condition. The variation of these items is too numerous to list but are items typically needed to respond to or treat the condition the kit is designed to address. By way of example, some general treatment materials for a first aid kit may include, anti-biotics, pain relievers, bandages, ointments, and wound cleaning items. Treatment items for a snake bit kit, opioid overdose kit, blood borne pathogen kit would be different and contains those items specific to addressing those particular conditions. Similarly, the plurality of video files will contain condition specific information corresponding to the condition the treatment kit is designed to address.

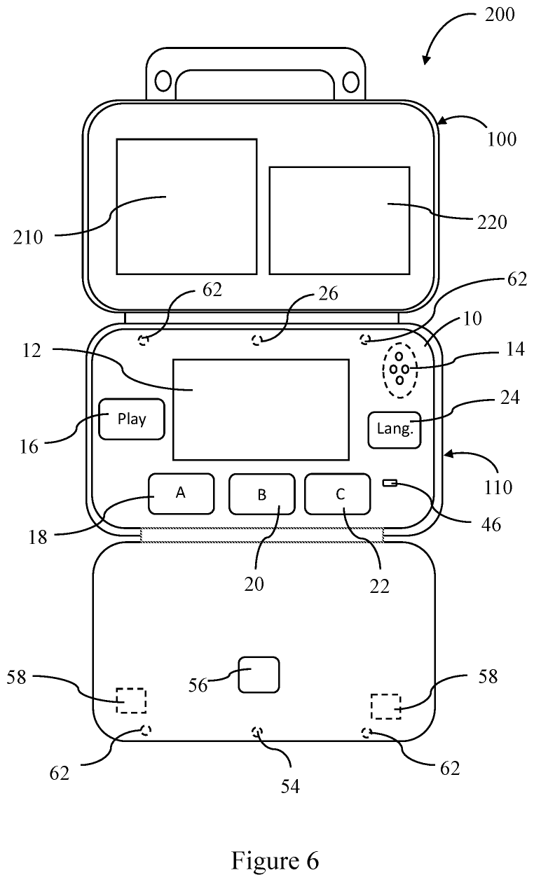

With reference to FIG. 6, and continuing reference to FIG. 5, the treatment kit 200 is illustrated where the cover 50 is in the open position, exposing the video monitor 12 and switches 16, 18, 20, 22, and 24.

The multi-media insert 10 above is designed to be easy to use. With reference to the embodiment illustrated in FIGS. 4, 5 and 6, a user will open the container 100 revealing the multi-media insert 10 in the lid of the container. The user will move the cover 50 to the open position by pulling on pull tab 64. When the cover 50 moves towards the open position, switch 26 activates and plays the programmed video file containing condition specific information sending audio content to the speaker and video content to the video monitor. The user may control the playback of the video using switch 16 to pause or replay the selected video file, or the user may select a different video file by selecting one of switches 18, 20, or 22. The user may select a different language for the audio content by using switch 24. If needed, the user may use a mobile device to scan the scannable code 56 to access substantially similar video files from the internet. Further the user may use their mobile device to tap near field communication chip 58 to dial emergency services or tap near field communication chip 60 to access and play substantially similar video files from the internet on the mobile device.

The following non-limiting examples are provided to illustrate certain embodiments of the invention.

Multi-media insert for opioid overdose conditions. The insert for the opioid overdose conditions includes a rapid response video file on emergency treatment procedures and a series of training video files on the instruction of administering overdose treatment medication, such as naloxone, to a subject. The insert has a cover and activates an embedded magnetic switch when the cover is opened. Upon activation a rapid response video file plays and repeats three times. The user is able to pause or replay the video at any time by pressing a play/pause button. A button is provided to switch the audio between English and Spanish. A training video button is provided to select and play any for the desired training video files on the video monitor. A scannable QR code is printed on the multimedia insert that links a mobile device to internet-based video files that are substantially similar to the rapid response video file and the training video file. A near field communication chip programmed for a mobile device to dial 9-1-1 is provided. The insert is sized to fit in the lid of a 10U plastic container.

Kit for opioid overdose condition. The kit for opioid overdose conditions includes the multi-media insert for opioid overdose conditions described above. The insert is located inside the lid of a 10U container. The kit is provided with personal protective equipment that includes gloves and a CPR barrier for the user. The kit has space for the addition of treatment medication such as naloxone.

Multi-media insert for basic first aid. The insert for the first aid kit includes a rapid response video file on identifying emergency situations and treatment videos files providing instructions on how to treat burns, basic illnesses, lacerations, fractures, allergic reactions, and bug bites in separate video files. The insert has a cover and activates an embedded magnetic switch when the cover is opened. Upon activation the rapid response video file plays and provides the user with virtual buttons on a touchscreen monitor that identify and link to each of the treatment video files. The user touches the screen to select the desired treatment video. A virtual button is provided on the touchscreen during video playback to pause or replay the video at anytime by pressing the virtual play/pause button. A button is provided to switch the audio between English and Spanish. A scannable QR code is printed on the multimedia insert that links a mobile device to internet-based video files that are substantially similar to the rapid response video file and the treatment video files. A near field communication chip programmed for a mobile device to dial 9-1-1 is provided. The insert is sized to fit in the lid of a 10U plastic container.

Kit for basic first aid. The kit for basic first aid includes the multi-media insert for basic first aid described above. The insert is located inside the lid of a 10U container. The kit is provided with personal protective equipment that includes gloves and a mask for the user. The kit is provided with treatment materials such as, gauze pads, adhesive bandages, roller bandages, antiseptic wipes, hydrogen peroxide, anti-biotic ointment, hydrocortisone crème, ibuprofen, acetaminophen, antihistamines, throat lozenges, cold medication, laxatives, tweezers, finger splint, cold pack, and an emergency blanket.

While only a few embodiments of the present invention have been shown and described, it will be obvious to those skilled in the art that many changes and modifications may be made thereunto without departing from the spirit and scope of the invention as described in the claims. All United States patents and patent applications, all foreign patents and patent applications, and all other documents identified herein are incorporated herein by reference as if set forth in full herein to the full extent permitted under the law.

What is claimed is:

1. A multi-media insert comprising:
a video monitor;
a speaker;
a switch;
a video controller having video file storage memory; and
a video file including video content and audio content having condition specific information for a specific condition stored in the video file storage memory,
wherein the video controller is in communication with the video monitor, the speaker, and the switch, and wherein the video controller plays the video content on the video monitor and plays the audio content on the speaker when the switch is activated.

2. The multi-media insert according to claim 1, further comprising a cover substantially covering the video monitor, wherein the cover is in communication with the switch and activates the switch when the cover is moved away from the video monitor.

3. The multi-media insert according to claim 2, wherein the cover is connected to the multi-media insert by a hinge, wherein the cover substantially covers the video monitor in a closed position and provides visual access to the video monitor when the cover is in the open position, and wherein the cover activates the switch when the cover is moved towards the open position.

4. The multi-media insert according to claim 1, wherein the switch is selected from the group consisting of a button-type switch, optical switch, magnetic switch, pressure switch, light sensor, motion sensor, and hinge sensor.

5. The multi-media insert according to claim 1, wherein the video monitor is a touchscreen video monitor.

6. The multi-media insert according to claim 1, further comprising:
a plurality of video files stored in the video file storage memory, wherein each of the plurality of video files includes video image content and video audio content having condition specific information for a specific condition, wherein each of the plurality of video files contain different condition specific information;
a plurality of switches in communication with the video controller, wherein at least one of the plurality of switches is programmed for the video controller to play at least one of the plurality of video files and provide the video image content to the video monitor and the audio content to the speaker when the at least one of the plurality of switches is activated.

7. The multi-media insert according to claim 1, wherein the at least one of the plurality of switches controls playback functions of the video file.

8. The multi-media insert according to claim 1, wherein the audio content contains audio content in more than one language.

9. The multi-media insert according to claim 6, wherein the audio content contains audio content in at least two languages, and wherein one of the plurality of switches is programmed to select one of the at least two languages when the at least one video of the plurality of videos is played on the video monitor and the speaker.

10. The multi-media insert according to claim 6, wherein one video file of the plurality of video files contains condition specific information for a first condition, and wherein a second video file of the plurality of video files contains condition specific information for a second condition, wherein one switch of the plurality of switches is programmed for the video controller to play the one video file and a second switch of the plurality of switches is programmed for the video controller to play the second video file.

11. The multi-media insert according to claim 1, wherein the condition is selected from the group consisting of drug overdoses, snake bites, allergic reactions, choking, CPR, cardiac arrest, heat related illnesses, burns, lacerations, bone fractures, disposal of medications, basic first aid, eye injuries, dental injuries, biohazardous spills, drowning and near drowning conditions, blood borne pathogen incidents, pet first aid, and food related illness.

12. The multi-media insert according to claim 10, wherein the first condition and the second condition are each selected from the group consisting of drug overdoses, snake bites, allergic reactions, choking, CPR, cardiac arrest, heat related illnesses, burns, lacerations, bone fractures, disposal of medications, basic first aid, eye injuries, dental injuries, biohazardous spills, drowning and near drowning conditions, blood borne pathogen incidents, pet first aid, and food related illness, wherein the first condition and second condition are different.

13. The multi-media insert according to claim 1, wherein the condition is opioid overdose treatment.

14. The multi-media insert according to claim 1, further comprising a code scannable by a mobile device that retrieves and plays an internet-based video file that has substantially the same video content and substantially the same audio content as the video file stored in the video file storage memory.

15. The multi-media insert according to claim 1, further comprising at least one near field communication chip, wherein the near field communication chip is programmed for a mobile device to dial emergency services.

16. The multi-media insert according to claim 1, further comprising at least one near field communication chip, wherein the near field communication chip is programmed for a mobile device to retrieve and play an internet-based video file that has substantially the same video content and substantially the same audio content as the video file stored in the video file storage memory.

17. The multi-media insert according to claim 1, wherein the multi-media insert is sized to fit in a lid of a container.

18. The multi-media insert according to claim 1, further comprising a power supply connected to the video controller and provides electrical power to the multi-media insert to run the video controller, video monitor, and speaker.

19. The multi-media insert according to claim 18, wherein the power supply is a rechargeable battery.

20. A treatment kit comprising:
a container having a lid;
treatment materials housed inside the container; and
a multi-media insert positioned on lid and within the container, the multi-media insert comprising:
a video monitor;
a speaker;
a switch;
a video controller having video file storage memory; and
a video file including video content and audio content having condition specific information for a specific condition stored in the video file storage memory, wherein the video controller is in communication with the video monitor, the speaker, and the switch, and wherein the video controller plays the video content on the video monitor and plays the audio content on the speaker when the switch is activated.
